(19) United States
(12) Patent Application Publication
(10) Pub. No.: US 2010/0304426 A1
(43) Pub. Date: Dec. 2, 2010

(54) Analytical Methods for Measuring Synthetic Progesterone

(76) Inventors: David OSBORNE, Fort Collins, CO (US); Paul WINKLER, Golden, CO (US)

Correspondence Address: GREENLEE SULLIVAN P.C., 4875 PEARL EAST CIRCLE, SUITE 200, BOULDER, CO, 80301, US

(21) Appl. No.: 12/787,495

(22) Filed: May 26, 2010

Related U.S. Application Data
(60) Provisional application No. 61/181,366, filed on May 27, 2009.

(51) Int. Cl. C12Q 1/02; G01N 33/48

(52) U.S. Cl. 435/29; 436/63

(57) ABSTRACT

Embodiments relating to methods, processes and systems for measuring progesterone are provided. In particular, methods permit measurement and quantification of synthetic and/or endogenous progesterone from a progesterone-containing blood fluid sample by measuring a progesterone carbon isotope ratio by mass spectrometry and calculating the fraction of synthetic progesterone in the sample from the isotope ratio. Also provided are methods of evaluating bioequivalence of a synthetic progesterone composition using any of the methods provided herein. In an embodiment, methods of precise measurements of plasma levels are described for detection of progesterone analytes such as total progesterone, endogenous animal progesterone, and synthetic progesterone. Correcting for fluctuations in endogenous progesterone levels following application of synthetic progesterone allows a significant reduction in the number of test subjects required to evaluate bioequivalence of a synthetic progesterone composition.

### CROSS-REFERENCE TO RELATED APPLICATIONS

This application claims benefit of U.S. Provisional Patent Application 61/181,366 filed May 27, 2009, which is hereby incorporated by reference to the extent not inconsistent herewith

### BACKGROUND OF THE INVENTION

In vivo hormone analysis and quantification is important as the number and frequency of hormone-replacement and other medical treatments using synthetic hormones increases. For example, progesterone is often prescribed with estrogen or estrogen-androgen therapy for treatment during or following menopause. Conventional methodologies for detecting and measuring progesterone are inadequate and imprecise. For example, present approaches for analyzing progesterone in patients taking synthetic progesterone cannot distinguish the exogenously administered synthetic progesterone from natural progesterone produced in the body. This deficiency can make it particularly difficult to understand and establish the interplay between synthetic and endogenous progesterone. It is currently unclear whether administration of synthetic progesterone affects endogenous production of progesterone. There is a need for better approaches and techniques for the measurement and analysis of progesterone. Accordingly, presented herein are embodiments including a variety of methodologies capable of measuring progesterone and distinguishing synthetic progesterone from endogenous progesterone.

Progesterone is formed in the ovary, testis, adrenal cortex, and placenta. Progesterone is a steroid hormone involved in the female menstrual cycle, pregnancy and embryogenesis. Endogenous (or native) levels of progesterone in females can be influenced by many factors including circadian rhythms, diet and environmental conditions, timing within the menstrual cycle, and artificial and natural changes in the body including those relating to the reproductive system, e.g., the life stages of menopause. The half life of progesterone in the circulation is reported to be only a few minutes. Thus, assay values for progesterone plasma levels in samples from a single individual can fluctuate widely within a day and between days. Further, if the individual such as a female is supplementing native levels of progesterone by taking synthetic progesterone for birth control or hormone replacement therapy, assay values for progesterone plasma levels can vary to an even greater extent, especially since treatment with synthetic progesterone may induce or alter the secretion of endogenous progesterone. In addition to intra-individual variability, there is recognition of significant variability of progesterone between individuals, including levels in populations such as in human females.

To better understand the role of progesterone in women's health and in mammalian biology in general, it is important to be able to detect and analytically separate native and synthetic steroid hormones. More specifically, in the development of healthcare products, it is desirable to detect and separate endogenous progesterone plasma concentrations from dosed synthetic progesterone plasma concentrations. A particular benefit of an embodiment of improved progesterone detection relates to the evaluation of the bioequivalence of orally administered synthetic progesterone products.

Synthetic progesterone commonly uses the steroid diosgenin as a starting material. Diosgenin is produced in relatively large amounts in the yam family such as from the genus Dioscorea. Progesterone derived from plant origin has a different abundance of the carbon isotope ¹²C relative to a heavier form, ¹³C, than progesterone derived from animal origin. This difference in carbon isotope ratios may be used as a basis for distinguishing endogenous progesterone from synthetic progesterone in blood fluid samples obtained from individuals taking synthetic progesterone.

### SUMMARY OF THE INVENTION

In an embodiment of the invention, an analytical method is disclosed that is capable of measuring and quantifying the level of synthetic progesterone in the presence of native (e.g., endogenous) progesterone. The method uses the difference in C¹² to C¹³ isotope ratios between native and synthetic progesterone to correct the measured total progesterone concentration for the contribution of native progesterone. The two major isotopes of the element carbon are ¹²C and ¹³C. The difference in these two forms of carbon is that the ¹²C atom has six protons and six neutrons in the nucleus while the ¹³C isotope has six protons and seven neutrons in the nucleus. The result of this difference is that ¹³C atoms have an atomic weight one mass unit higher than that of ¹²C atoms. This mass difference can be detected by a mass spectrometer, which forms the basis of this invention. Because the naturally occurring frequency of ¹³C atoms is 1.10% of ¹²C atoms, it is statistically expected, therefore, for every 100 ¹²C atoms present, there will be approximately 1 ¹³C atom. The molecular formula for progesterone is C₂₁H₃₀O₂ resulting in a molecular weight of 314 atomic mass units (amu). Due to the natural abundance of ¹³C, it is expected that for approximately every five molecules of progesterone, one of the carbon atoms will be ¹³C instead of ¹²C resulting in a molecule that has a molecular weight of 316 amu. The ion observed for progesterone using this method is at mass 315. There is also an ion observed at mass 316 that arises from those molecules containing one ¹³C atom. It is possible to measure the intensity of the signal from the ion at mass 315 and 316. A ratio of these two signal intensities is a measure of the relative amount of ¹²C to ¹³C in the progesterone. Plants tend to have a higher abundance of ¹³C atoms present in their molecules compared to animals and therefore a different ratio of ¹²C/¹³C is expected for plant derived hormones compared to animal derived hormones. Comparing the signal associated with mass 315 to mass 316, which is the ¹²C/¹³C ratio, for human derived progesterone demonstrates a ratio of approximately 6.49. Comparing the signal of mass 315 to mass 316 for plant derived progesterone demonstrated a ratio of approximately 6.33. In an embodiment of an analytical method of the invention, upon interpolation the observed carbon isotope ratio values vary continuously from approximately 6.33 for zero native progesterone (i.e., all of the progesterone corresponds to synthetic progesterone) to approximately 6.48 for zero synthetic progesterone (i.e., all of the progesterone corresponds to endogenous progesterone). Accordingly, by measuring the carbon isotope ratio of progesterone, it is possible to determine how much of the signal is attributable to the endogenous progesterone and/or synthetic progesterone. In an aspect, the ratio values for all synthetic and for all natural can vary from the values provided herein, such as by depending on the source of progesterone and instrumentation and instrumentation parameters used. Optionally, the procedure may further involve establishing “baseline” carbon isotope ratio values for an assay procedure. Accordingly, the relevance of the processes disclosed herein is not a particular value for the carbon isotope ratio, but instead the recognition that there is a difference between the natural and synthetic carbon isotope ratio of progesterone. In an aspect, the difference is between about 0.15 and 0.21 for the experimental conditions outlined herein.

The discovery and development of the superior approaches for analyte detection and measurement through embodiments of the invention now make it possible to provide quantitative information, such as for synthetic progesterone in the presence of endogenous progesterone. This also translates into a major advance in assessments of bioequivalence for products including therapeutics. Embodiments of the invention provide the opportunity to gain insight into the interplay of synthetic and native hormones. Lack of this insight has limited the advancement and approval of therapeutic products. By solving this analytical method problem, new hormone products can be developed and bioequivalence can be more readily evaluated and established for synthetic and semi-synthetic hormones, including sex steroids such as progesterone.

Due to embodiments of the present invention which provide improved methods of measuring progesterone analytes and distinguishing the sources of progesterone, there has been an important advance in the understanding of the biology of synthetic progesterone treatment. Because of improved analytical techniques, it is now recognized that exposure to synthetic progesterone can have a significant effect on the plasma levels of endogenous progesterone. It is possible that the administration of synthetic progesterone induces the production of endogenous progesterone.

It can be particularly difficult to analyze the pharmacokinetics of progesterone in situations where application of synthetic progesterone can, in turn, up-regulate endogenous progesterone production. Conventional methods quantify total progesterone and do not distinguish between endogenous and synthetic progesterone. This inability to distinguish between the different progesterone sources (endogenous versus synthetic) in the circulating blood can lead to increases in the variability of a measured pharmacokinetic parameter, making it difficult to establish good pharmacokinetic parameters for synthetic progesterone. Increase in variability of a pharmacokinetic parameter also makes establishing bioequivalence of a progesterone composition with another composition more difficult, with larger variations in a measured or calculated pharmacokinetic parameter requiring correspondingly larger sample sizes to establish statistical validity.

For example, any of the processes disclosed herein may be used to evaluate and/or establish bioequivalence of generic formulations of PROMETRUIM® synthetic progesterone (Solvay Pharmaceuticals, Inc., Marietta, Ga.). In particular, any of the synthetic progesterone disclosed herein may be obtained from a starter material isolated from plants, such as diosgenin isolated from yams in the genus Dioscorea.

In an embodiment, the invention provides a method of measuring a progesterone analyte in a blood fluid sample, such as by providing a blood fluid sample and introducing a progesterone component obtained from the sample to a mass spectrometer. The progesterone component comprises at least a portion of all the progesterone in the sample, or it optionally comprises all the progesterone in the sample. For example, the progesterone in the sample may be appropriately diluted or concentrated to provide a desired amount to the mass spectrometer to ensure maximum accuracy and sensitivity when performing mass spectrometry. The mass spectrometer provides a measure of the carbon isotope ratio, such as a ¹²C/¹³C isotope ratio (or correspondingly, the inverse of the ¹²C/¹³C isotope ratio, ¹³C/¹²C). The isotope ratio is used to calculate a fraction of synthetic progesterone of the introduced progesterone component, thereby measuring the progesterone analyte in said sample.

In an aspect, the method further comprises obtaining the blood fluid sample from a subject and isolating the progesterone component from the sample. The blood sample may be obtained by an intravenous blood draw, such as a blood draw at selected times after administration of a progesterone composition to the subject.

In an aspect, any of the methods disclosed herein relates to any two of synthetic, natural and total progesterone being measured, such as synthetic progesterone and at least one more of endogenous and total progesterone, in either the progesterone component, the progesterone in the sample, or the whole-body. In another aspect, any of the methods disclosed herein relates to the measurement of total progesterone and the calculation of synthetic progesterone using the isotope ratio to correct for that fraction of the measured signal that arises from natural progesterone in either the progesterone component, the progesterone in the sample, or the whole-body.

In an embodiment, the method relates to calculating a concentration or amount of synthetic progesterone and/or endogenous progesterone in the sample.

In an embodiment, any technique as would be understood in the art is optionally used to introduce progesterone, such as substantially purified progesterone, to the mass spectrometer. In a preferred embodiment, the progesterone is isolated or processed by liquid chromatography. In one embodiment, the mass spectrometer is a liquid chromatography-tandem mass spectrometer.

In an aspect the blood fluid sample is plasma, serum or whole blood. In an aspect, the sample is obtained from a mammal, such as a human.

In one embodiment, the method further comprises administering synthetic progesterone to an individual prior to obtaining the blood fluid sample, such as synthetic progesterone obtained from a plant source. In an aspect of this embodiment, the synthetic progesterone is from yam of the genus Dioscorea, and in particular made from diosgenin obtained from yam. In an aspect, the synthetic progesterone is PROMETRUIM® progesterone (pregn-4-ene-3,20-dione) by Solvay Pharmaceuticals, Inc. (Marietta, Ga.) or a generic thereof.

In an aspect, any of the methods provided herein relate to a calculating step that comprises quantification of one or more of synthetic progesterone, natural progesterone and total progesterone, wherein the quantification is capable of detecting synthetic progesterone, natural progesterone or total progesterone in a blood fluid sample at a level that is less than or equal to 0.1 ng/ml, 0.01 ng/mL, or from about 0.01 ng/mL to 0.1 ng/mL. In an aspect, any of the methods provided herein relate to a calculating step that comprises quantification of total progesterone and calculation of synthetic or natural progesterone from the total progesterone quantification, wherein the quantification is capable of detecting synthetic progesterone, natural progesterone or total progesterone in a blood fluid sample at a level that is less than or equal to 0.1 ng/ml, 0.01 ng/mL, or from about 0.01 ng/mL to 0.1 ng/mL. In a particular embodiment, the blood fluid is plasma. In a particular embodiment, the plasma is human plasma.

In one embodiment, any of the methods provided herein further comprise generating a calibration curve that provides a concentration of total progesterone, a fraction of synthetic or natural progesterone for a measured carbon isotope ratio for a defined fraction of synthetic progesterone in a progesterone-containing sample, such as ¹³C/¹²C isotope ratio. The calibration curve may be generated for a given source material, e.g., that corresponding to each batch or lot of synthetic progesterone used on the subjects. The calibration curve is optionally updated continually or periodically as part of a quality control scheme.

For example, the calculating step optionally relates to calculating the fraction of synthetic progesterone in the sample by providing an isotope ratio curve that defines the fraction of synthetic progesterone for the measured ¹³C/¹²C progesterone isotope ratio, and calculating a synthetic progesterone level from the fraction as determined by the measured isotope ratio and the isotope ratio curve.

Also provided are methods of quantifying a progesterone analyte in a subject by obtaining a blood fluid sample from the subject, isolating a progesterone component from the sample, introducing the progesterone component to a mass spectrometer, measuring a carbon isotope ratio of the progesterone component and calculating from the isotope ratio the amount of progesterone analyte in the sample, thereby quantifying the progesterone analyte in the subject. In an embodiment, the subject is provided progesterone, such as synthetic progesterone, before the blood fluid sample is obtained. The quantification optionally relates to determination of circulating progesterone analyte level or concentration in whole blood.

In an aspect, the method is performed on a plurality of subjects, such as repeating the quantification for a plurality of subjects and calculating a pharmacokinetic parameter for the plurality of subjects from the measured isotope ratios and calculating a statistical parameter for the pharmacokinetic parameter. In an embodiment of this aspect, the statistical parameter is reduced compared to a corresponding statistical parameter calculated using a conventional progesterone quantifying method. In an embodiment, the reduction is by at least 20%, at least 50%, or from about 20% to 80%. In an embodiment, the statistical parameter relates to a progesterone analyte that is synthetic progesterone.

In an embodiment, the statistical parameter is a coefficient of variation, standard deviation, standard error of the mean, or a range. In an embodiment, the statistical parameter is any parameter that, directly or indirectly, is useful in evaluating bioequivalence of a progesterone composition against another progesterone composition.

In an aspect, the pharmacokinetic parameter is selected from the group consisting of Cₘₐₓ, Tₘₐₓ, half-life, clearance time, rate of absorption, and AUC (“area under the curve”).

In any of the methods provided herein, progesterone is provided to an individual, and the provided progesterone results in an increase in endogenous progesterone in a blood fluid sample. In an embodiment, the provided progesterone induces production or alters the distribution or metabolism of endogenous progesterone.

In an embodiment, the progesterone analyte corresponds to synthetic progesterone.

In another embodiment, the progesterone component comprises synthetic and endogenous progesterone.

In an aspect, the invention is the use of any of the methods provided herein to evaluate bioequivalence of one synthetic progesterone-containing compound to a second synthetic progesterone-containing compound, such as a follow-on generic compound of a brand-name progesterone compound, such as PROMETRIUM® progesterone.

In another embodiment, the invention is a method of evaluating bioequivalence of a synthetic progesterone composition, such as by administering the composition to a plurality of subjects, obtaining a blood fluid sample from the subjects after the administering step, quantifying synthetic progesterone in the sample by measuring a ¹³C/¹²C progesterone carbon isotope ratio (e.g., ¹³C/¹²C or ¹²C/¹³C), and calculating a synthetic progesterone pharmacokinetic parameter from the isotope ratio.

In an aspect, bioequivalence is evaluated by comparing the calculated pharmacokinetic parameter against a corresponding pharmacokinetic parameter from a second synthetic progesterone-containing compound. Optionally, the pharmacokinetic parameter is one or more of Cₚᵣₑ, Cₘₐₓ, Tₘₐₓ, Cₗₐₛₜ and AUC.

In embodiments, an advantage related to the methods provided herein is that bioequivalence may be evaluated, and more particularly established, with a lower number of subjects compared to methods that do not address whether progesterone in the blood sample may also have endogenous progesterone that is upregulated in response to application of synthetic progesterone. Accordingly, also provided are methods wherein bioequivalence is evaluated using a subject number that is at least 20%, or at least 50% lower than the number required using a conventional progesterone-quantifying assay that does not distinguish between synthetic and natural progesterone. This decrease in required subject number is related to the ability to decrease variability in the measured synthetic progesterone (e.g., a reduction in the statistical parameter of the synthetic progesterone pharmacokinetic parameter) by accounting for endogenous progesterone in the sample.

In an embodiment, the method further comprises calculating a statistical parameter for the pharmacokinetic parameter, wherein the statistical parameter is reduced by at least 20% compared to a corresponding statistical parameter obtained using a conventional progesterone-quantifying assay. Although any statistical parameter of interest may be reduced, in an aspect the statistical parameter is standard deviation, standard error of the mean, coefficient of variation, or a range.

In an aspect, the sample is obtained between 1 hour and 8 hours after the synthetic progesterone is introduced to the subject.

In an aspect, any of the methods presented herein relate to a carbon isotope ratio that is the ratio of ¹³C to ¹²C or ¹²C to ¹³C of an analyte. In an embodiment, the analyte is progesterone.

In an embodiment, any of the methods provided herein relate to synthetic progesterone that is PROMETRUIM® progesterone (pregn-4-ene-3,20-dione) by Solvay Pharmaceuticals, Inc. (Marietta, Ga.).

Without wishing to be bound by any particular theory, there can be discussion herein of beliefs or understandings of underlying principles or mechanisms relating to the invention. It is recognized that regardless of the ultimate correctness of any explanation or hypothesis, an embodiment of the invention can nonetheless be operative and useful.

### BRIEF DESCRIPTION OF THE DRAWINGS

FIG. 1 is an isotope ratio curve of progesterone carbon isotope ratio as a function of the fraction of natural progesterone that is useful in measuring the fraction of natural (and thereby synthetic) progesterone in a progesterone-containing sample by mass spectrometry.

FIG. 2A-2D provides a time course of total progesterone concentration and synthetic progesterone concentration for four individuals provided with a single dose of synthetic progesterone at time, t=0.

FIG. 3 is an overlay of the time course of synthetic progesterone of the individuals of FIG. 2.

FIG. 4 plots the average of the synthetic progesterone and associated standard deviation from FIG. 3 (n=4).

FIG. 5 is a time course plot of the means of each of total, synthetic and endogenous progesterone after administration of synthetic progesterone at t=0 (n=4).

FIG. 6 is a time course of total progesterone for blood fluid samples from four subjects who were administered synthetic progesterone at t=0.

FIG. 7 is a time course of synthetic progesterone for blood fluid samples from four subjects who were administered synthetic progesterone at t=0.

FIG. 8 is a time course of endogenous progesterone for blood fluid samples from four subjects who were administered synthetic progesterone at t=0.

FIG. 9 is a time course of the average total progesterone for subjects administered synthetic progesterone at t=0 (n=4).

FIG. 10 is a time course of the average synthetic progesterone for subjects administered synthetic progesterone at t=0 (n=4).

FIG. 11 is a time course of the average endogenous progesterone for subjects administered synthetic progesterone at t=0 (n=4).

### DETAILED DESCRIPTION OF THE INVENTION

An aspect of the present invention provides the capacity to distinguish between endogenous and synthetic progesterone in a progesterone-containing sample that may contain both endogenous and synthetic progesterone. In contrast, “conventional” progesterone quantifying methodologies do not provide any distinction, but instead suffer the disadvantage of providing only an indication of total progesterone (e.g., both synthetic and endogenous progesterone). “Natural” and “endogenous” progesterone are used interchangeably to refer to progesterone that is produced by the subject, in contrast to “synthetic” progesterone that is administered or introduced to the subject, such as progesterone that is isolated from a plant source. In a broad sense, as used herein synthetic progesterone refers to progesterone from any of a variety of sources that have an isotope ratio that is detectably different from progesterone that is endogenously produced by the individual.

As used herein, “progesterone analyte” refers to a material whose quantification provides information about progesterone in a sample or individual. For example, a progesterone analyte may be one or more of synthetic, endogenous or total progesterone. A particular example of a progesterone analyte is synthetic progesterone. Alternatively, a progesterone analyte may instead be a progesterone precursor, metabolite or other compound that is related to progesterone including, but not limited to, pregnenolone, 160H-progesterone, phytosterols, plant sterols, or phytostanols.

“Progesterone component” refers to at least a portion of all the progesterone in a sample that is introduced to a mass spectrometer. In an aspect, substantially all or all of the progesterone in the sample is introduced to the mass spectrometer. “Substantially” is used herein to refer to at least 90%, at least 95%, or at least 98% of the absolute value. In an aspect, only a portion of all the progesterone is introduced to the mass spectrometer, such as a known fraction of the total amount to permit quantitative analysis so that absolute progesterone levels and/or concentrations may be calculated. The progesterone component introduced to the mass spectrometer may have a synthetic fraction that corresponds to the synthetic fraction of the blood fluid sample which, in turn, may correspond to the synthetic fraction of progesterone in the circulating blood in the individual from whom the blood sample is obtained.

When referring to fraction of synthetic or fraction of endogenous, “fraction” refers to the fraction of synthetic and/or endogenous progesterone components in the progesterone component. In embodiments herein, these fractions are determined by measuring carbon isotope ratios by mass spectrometry.

“Measuring” is used broadly to refer to information useful in distinguishing between the various progesterone analytes, such as distinguishing synthetic progesterone from endogenous or natural progesterone. In an aspect, measuring refers to determining the fraction of synthetic progesterone in a progesterone-containing sample. In an aspect, measuring refers to quantifying the level of synthetic progesterone in a progesterone-containing sample. Quantifying refers to either an absolute level or a concentration, either in the sample or from the individual from whom the blood fluid sample is obtained.

“Sample” refers to a portion of material such as a blood fluid sample obtained from the individual for which progesterone measurement is desired. In an aspect, the individual is a human. The sample may range from whole blood or a suspected progesterone-containing component thereof, such as plasma, platelet free plasma, or serum.

“Isotope ratio” refers to the ¹³C/¹²C (or correspondingly ¹²C/¹³C) isotope ratio of progesterone. In a particular example, the ratio is determined by MS and it makes no difference to the methods provided herein whether the ratio measured or used is ¹³C/¹²C or ¹²C/¹³C, as determination of one defines the other. Accordingly, both ratios are encompassed by the term “isotope ratio”.

“Pharmacokinetic parameter” refers to a parameter useful for evaluating a compound's pharmacological profile, such as progesterone, that has been administered to an individual. Examples of key pharmacokinetic parameters include, for example, area under the curve (AUC), peak concentration (Cₘₐₓ), time to peak concentration (Tₘₐₓ), and absorption lag time (t_{lag}). In an aspect, the pharmacokinetic parameter is a parameter useful for establishing bioequivalence. Accordingly, a pharmacokinetic parameter may be selectively determined over a period of time, ranging from prior to progesterone administration to many hours after progesterone administration, and may reflect a time course of progesterone in circulating blood.

“Statistical parameter” refers to a statistical measure of a pharmacokinetic parameter obtained from a plurality of individuals whose progesterone analyte is measured. For example, the statistical parameter may provide a measure of the distribution of the measured pharmacokinetic parameter and may be useful in determining whether or not an administered progesterone composition has a pharmacokinetic parameter value that is not statistically different from another progesterone composition. The definition of statistical difference may be defined a priori, such as in accordance with a U.S. FDA accepted definition for establishing bioequivalence or applicable standard or regulation elsewhere. For example, bioequivalence may be established by if the 90% confidence interval of one or more pharmacokinetic parameters of a test compound is within a percentage range of the reference compound, such as within 80% to 125%. Accordingly, a statistical parameter may be any parameter useful in establishing a confidence interval, such as a confidence level of 80%, 90% or 95%, for example. In an aspect, any of the methods provided herein permit statistical achievement of a defined confidence interval for synthetic progesterone with a lower sample size by accounting for variations in endogenous progesterone.

As used herein, “bioequivalence” refers to the United States Federal Drug Administration definition that, “Bioequivalent drug products show no significant difference in the rate and extent of absorption of the therapeutic ingredient” and as provided by 21 U.S.C. §355(j)(8) and federal regulatory interpretation thereof (e.g., 21 CFR 320 et seq.). The term can also relate to the contexts of scientific analytical research, pharmaceutical product development, and regulatory systems in jurisdictions other than the United States.

A “synthetic progesterone composition” refers to a material that is capable of providing progesterone to a subject administered the composition, such as by oral ingestion or transdermal application. In an aspect, the composition comprises progesterone obtained from a plant source, such as from yam, for example. Alternatively, the composition contains a material that when subject to natural biological processes such as enzymatic activity, the material yields progesterone or a progesterone pre-material that is capable of being processed into progesterone. A functional definition of such materials is that the synthetic progesterone has a carbon isotope ratio (e.g., ¹³C/¹²C or ¹²C/¹³C) that is different from endogenous progesterone produced in the individual to whom the material is provided.

The invention may be further understood by the following non-limiting examples. All references cited herein are hereby incorporated by reference to the extent not inconsistent with the disclosure herewith. Although the description herein contains many specificities, these should not be construed as limiting the scope of the invention but as merely providing illustrations of some of the presently preferred embodiments of the invention. For example, thus the scope of the invention should be determined by the appended claims and their equivalents, rather than by the examples given.

### Example 1

### General Analytical Methodology

A definitive low-level LC/MS/MS analytical method to determine the concentrations of synthetic progesterone in human plasma is described. Instrumentation used in this example includes an Applied Biosystems Q-Trap 4000 system using Analyst® 1.4.2 software, Shimadzu LC-20AD HPLC pumps and a LEAP HTC PAL Autosampler. As understood by a person skilled in the art, various similar or equivalent pieces of instrumentation can be used to perform this method. It is also recognized that improved instrumentation can be introduced utilizing equivalent or similar principles of analysis to perform this method similarly or better by being faster, more sensitive, more accurate or more robust relative to the instrumentation used herein.

One skilled in the art will acknowledge that the following instrument parameters (Table 1), while nominally optimized for the method, will function equivalently well when varied, such as a variation up to 50%, with some parameters such as injection volume and flow rate being doubled or tripled without negatively impacting the method, especially if compensating changes are made in other instrument parameters. Table 1 provides representative parameters for HPLC (that isolates progesterone from a progesterone-containing sample) and subsequent MS that determines ¹³C/¹²C progesterone isotope ratio.

The Waters 2.1 mm diameter, 250 mm long, XBridge BEH130 C18 3.5 μm HPLC column specified in Table 1 may be used with the method disclosed herein. One skilled in the art will acknowledge that HPLC columns having similar packing material and similar particle size and column length can provide equivalent results. Likewise improved columns are constantly being introduced that can provide equivalent results. Column length, packing particle size, run times and the particular gradient used all influence the retention time of progesterone. These parameters can be modified in such a way that similar or equivalent results can be obtained despite dramatically changing each of these parameters in a mutually compensating manner. The pump gradient listed in Table 2 is complimentary to the instrument parameters listed in Table 1 and provides a progesterone retention time of about 13.6 minutes, but one skilled in the art can provide other suitable gradients for this method.

A calibration curve provides for quantification of progesterone and subsequent quantification of the progesterone isotope ratio. One suitable method for generating a suitable calibration curve is to prepare eight calibration solutions for analysis. Standards used for the solution preparation are made fresh every day until standard stability is established. These solutions are prepared by adding the indicated volume of progesterone dilution standard to a 10 mL volumetric flask (Table 3), adding in the indicated volume of internal standard (IS) dilution standard A and bringing to volume with HPLC water.

The final internal standard concentration is 15 ng/mL for each calibration solution. Calibration curves are prepared by adding 500 μL of each calibration solution (see Table 3) into 500 μL of blank plasma. Two calibration curves, including a blank and a zero sample, are prepared in wells that bracket the samples to be prepared. The zero sample is prepared by adding 500 uL of internal standard dilution standard B to 500 uL of blank plasma. The blank sample is prepared by adding 500 uL of HPLC water to 500 uL of blank plasma.

System suitability should be verified by generating a calibration curve at the beginning and end of an analytical sequence. Correlation coefficients (R²) should have a minimal acceptable value, such 0.95 and all quality control samples must be within a specified range of the nominal concentration, such as ±15%. Additional system suitability criteria will be apparent to one skilled in the art.

Low, mid, and high quality control samples can be prepared by spiking blank plasma following the spiking procedure for the level 2, level 5, and level 7 calibration solutions (Table 3).

Isotopic ratio standards include a natural isotopic ratio standard and a synthetic isotopic ratio standard. The natural isotopic ratio standard is prepared by adding 500 μL of internal standard dilution standard B to each of two 500 μL pregnant female plasma samples. After preparation, the two extracts are combined into one sample and analyzed to determine the natural progesterone ratio. The synthetic isotopic ratio standard is prepared by adding 500 μL of calibration solution 6 to 500 μL of blank plasma. Take two 25 ng/mL samples of this mixture. After preparation, the two extracts are combined into one sample and analyzed to determine the synthetic isotopic ratio.

Samples are prepared by placing a 96-well Oasis HLB plate on the vacuum manifold. Rinse each well to be used with 500 μL methanol. Apply enough vacuum to get drop-wise flow through the SPE beds. Rinse each well to be used with 500 μL water. Apply enough vacuum to get drop-wise flow through the SPE beds.

Vent the vacuum from the manifold. Transfer 500 μL of calibration solution into the wells that will be used for the calibration curves and the synthetic isotopic ratio standards. Transfer 500 μL of internal standard dilution standard B into the wells that will be used for zero, analytical samples, and the natural isotopic ratio standards. Transfer 500 μL of HPLC water into the wells that will be used for the blank samples. Transfer 500 μL of specified plasma into wells on the plate. Apply vacuum to the manifold to start elution of the sample at a flow rate of approximately 1 mL/minute. After the sample is completely eluted through the HLB bed, rinse with 500 μL water. Rinse the HLB bed with 500 μL 5% methanolic formic acid. Remove the plate from the manifold and place a 96-well collection plate in the bottom of the manifold. Place the extraction plate back on the manifold and add 500 μL of methanol to each sample well.

Gradually increase the vacuum until some elution begins. The vacuum must not be increased too rapidly or some wells will not be properly eluted. The elution of the bed may be viewed through the glass viewing window on the front of the manifold. Remove the sample plate and the collection plate from the manifold. Place the collection plate on the Zipvap concentrator or equivalent and concentrate the extracts to dryness. The Zipvap temperature is set to 40 ° C. and the nitrogen flow is set to 15 psi.

Reconstitute the samples by the addition of 100 μL of 50:50 water:acetonitrile:0.1% formic acid. Swirl the samples on the orbital shaker at 150 rpm for 5 minutes to fully dissolve. Transfer both pregnant female plasma extracts to a low volume insert autosampler vial. Transfer two of the three 25 ng/mL extracts from the first curve to a low volume insert autosampler vial.

Analyze the pregnant female plasma isotopic ratio standard seven consecutive times. Analyze the synthetic isotopic ratio standard at least seven consecutive times. Analyze the plate beginning with the first calibration sample. Using the data from the seven injections of pregnant female plasma calculate the average area for the progesterone transition and for the progesterone isotope transition. Calculate the ratio by dividing the average area of the progesterone peak by the average area of the isotope peak. This is the natural progesterone ratio. Using the data from the seven injections of the Synthetic Standard calculate the average area for the progesterone transition and for the progesterone isotope transition. Calculate the ratio by dividing the average area of the progesterone peak by the average area of the isotope peak. This is the synthetic progesterone ratio.

The isotope correction calculation is used to find the fraction of synthetic progesterone in the total progesterone signal, using Equation I:

S=(B−R)/(B−A) Equation I,

wherein: S=Fraction of signal from synthetic progesterone; B=Isotopic ratio from natural progesterone; A=Isotopic ratio from synthetic progesterone; and R=Observed isotopic ratio from the sample.

To correct the observed concentration of progesterone in the sample for the natural contribution, multiply the measured concentration by S, as shown in equation II:

Cₛ=Cₜ*S Equation II,

wherein: Cₛ=Concentration of synthetic progesterone; and Cₜ=Concentration of progesterone measured in the plasma.

Isotope ratio calibration to determine fraction of natural and/or synthetic progesterone in a progesterone-containing sample: Briefly, the isotope ratio is determined seven times for each sample and an average isotope ratio calculated. The isotope ratio is used to correct for natural progesterone in a progesterone sample that may contain both natural and synthetic progesterone.

Plasma is obtained from pregnant women (PFP) in the third trimester of pregnancy. This plasma should contain the highest concentration of progesterone. Extractions are from 1 mL of PFP and 1 mL of male human plasma that is spiked with 50 ng/mL synthetic progesterone. Sample extraction utilizes Oasis™ HLB solid phase extraction. Each extract is analyzed seven times on two different days (day 1 and day 2) and the isotope ratios remain unchanged, with an isotope ratio of 6.33±0.02 (SD) for male plasma spiked with synthetic progesterone and 6.49±0.05 (SD) for PFP. In other words, the isotope ratio for synthetic progesterone is 6.33 and for natural progesterone the isotope ratio is 6.49. The isotope measurement is stable as the average ratio is unchanged on both days of analysis. A 1:1 mixture of the natural and synthetic extracts is analyzed and the isotopic ratio of 6.40 is in good agreement with the expected value of 6.41 (for a linear relationship).

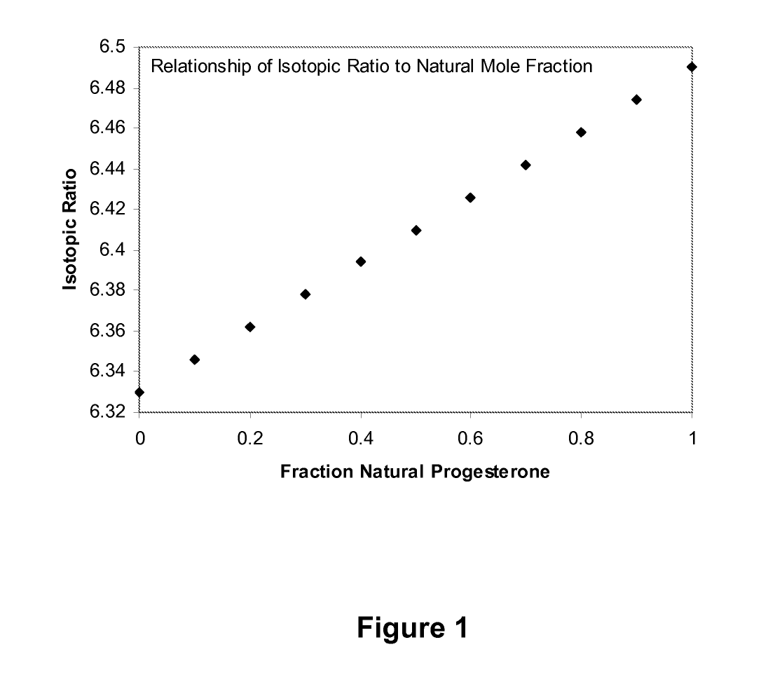

Assuming the isotopic ratio varies linearly with the fraction of natural progesterone, the isotopic ratio may be used to calculate the fraction of natural progesterone in the sample. Once the fraction of natural progesterone is known, it may be subtracted from the total amount of progesterone measured to determine the fraction or amount of synthetic progesterone. FIG. 1 is an isotope ratio curve that plots the relationship between fraction of natural progesterone and isotopic ratio. Accordingly, by measuring the isotopic ratio of progesterone (e.g. ¹²C/¹³C or ¹³C/¹²C) the fraction of natural (and, thereby, synthetic) progesterone can be calculated. This technique addresses the concern that, for example, natural or endogenous progesterone may change with synthetic progesterone treatment, thereby confounding statistical analysis of the pharmacokinetic parameters of the synthetic progesterone.

Preliminary chromatographic peak analysis of sample extract of PFP indicates the sample has sufficient signal to noise to accurately measure the isotopic ratio of progesterone. The observed peaks are similar to that obtained for samples having a progesterone concentration of about 25 ng/mL and the method exemplified herein is linear from at least 1 ng/mL to 500 ng/mL.

### Example 2

### Isotope Ratio (¹²C/¹³C) of Progesterone to Determine Synthetic Progesterone Levels

Synthetic and natural progesterone have different ¹³C to ¹²C isotope ratios. Synthetic progesterone made from yam extract has a lower ¹²C/¹³C ratio. A LC/MS/MS method provides a quantitative measure of either or both synthetic and endogenous progesterone in a sample potentially containing both components by measuring the carbon isotope ratio and comparing it against a carbon isotope ratio curve, such as one similar to that provided in FIG. 1 or from an equation obtained from standards containing known fractions of synthetic/natural progesterone.

Subjects are provided with progesterone soft gel cap (Prometrium®). An analytical methodology, as outlined in Example 1, is capable of distinguishing between endogenous (e.g., “natural”) progesterone from synthetic (e.g., administered) progesterone. Such an analytic technique can be used to reduce patient to patient variability in detected progesterone after application of synthetic progesterone, particularly in those patients where synthetic progesterone administration leads to stimulation of endogenous progesterone production.

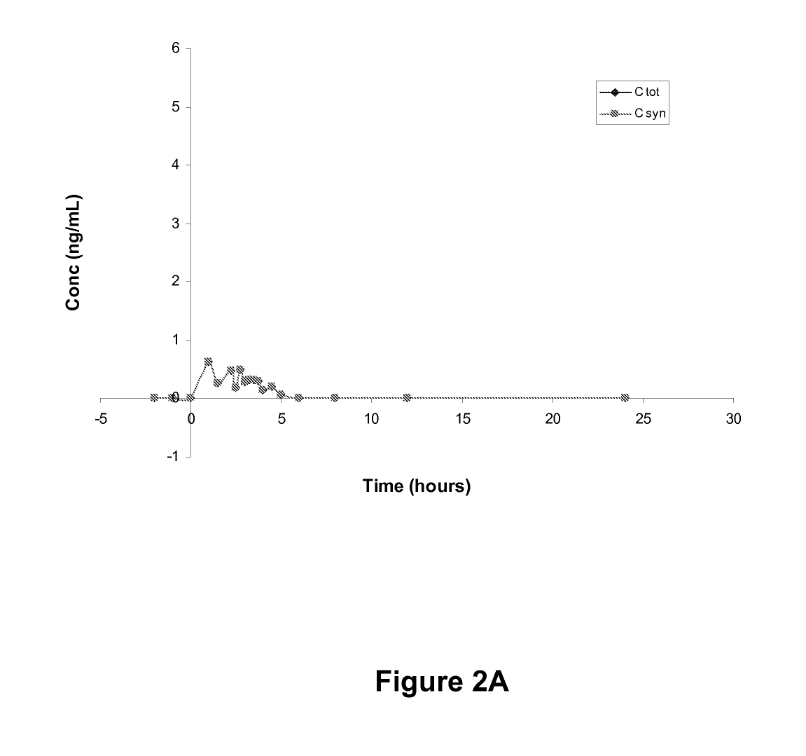

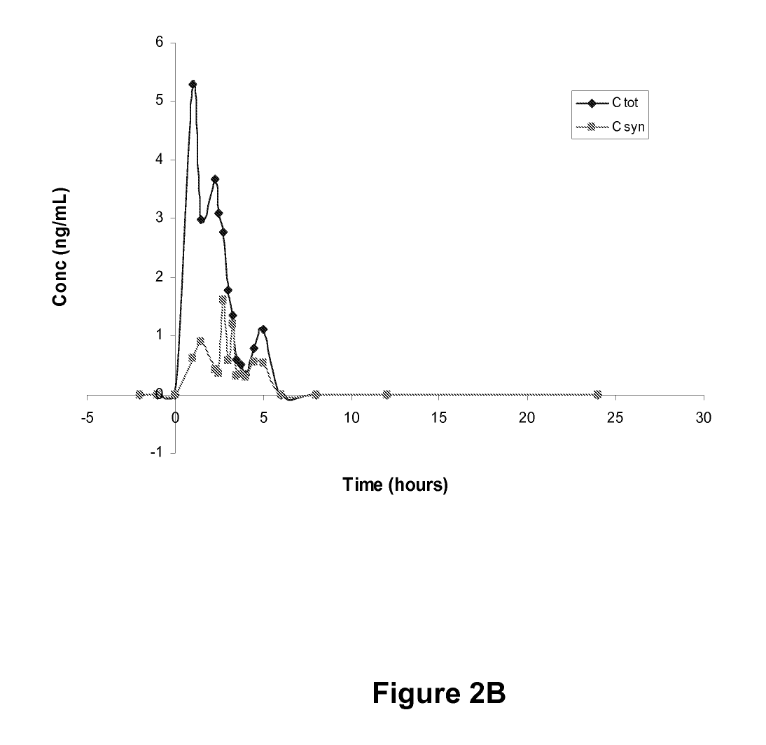

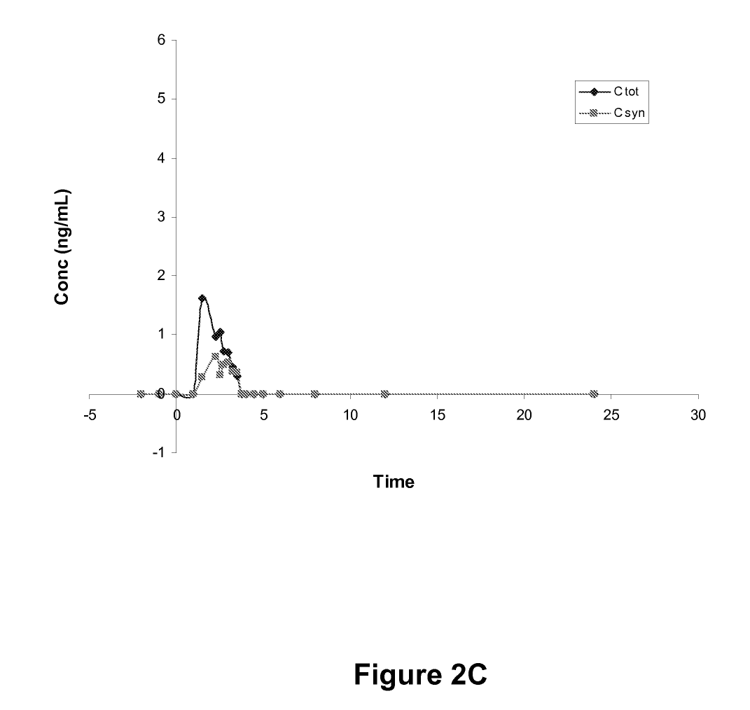

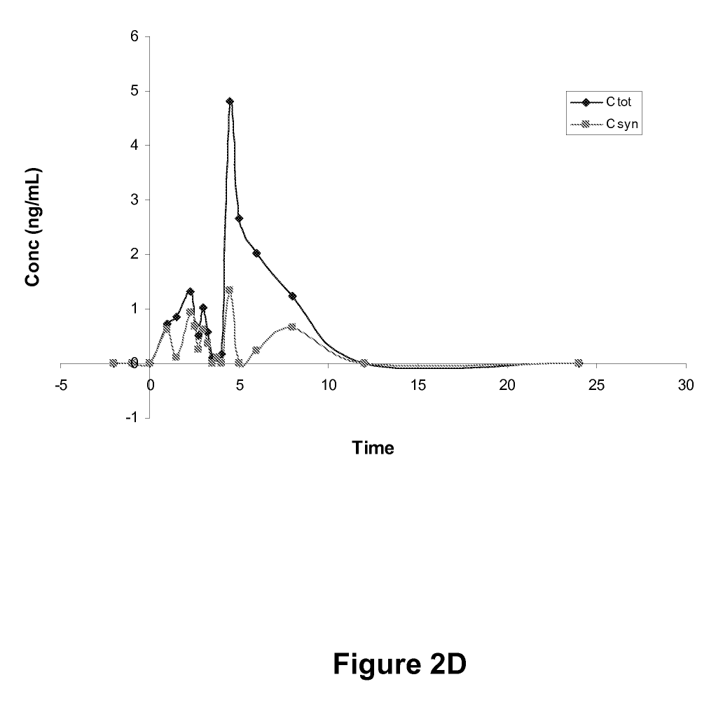

Preliminary results of progesterone concentration as a function of time for four different subjects are provided in FIG. 2A-2D. Total (Cₜₒₜ) and synthetic (C_{syn}) progesterone concentrations are plotted as a function of time, with synthetic progesterone (200 mg) administration (oral) at t=0 h. Preliminary indications are that detected progesterone levels are rather low and that dosed progesterone stimulates endogenous progesterone production (see, e.g., FIGS. 2B-2D). One portion of the patient population did not provide a quantifiable progesterone signal, suggesting the relevant progesterone response is less than 0.1 ng/mL, or that the time frame of progesterone increase was before the earliest sample collection time point of one hour. In addition, data presented herein are for fasted post-menopausal women. Fasting can affect progesterone uptake.

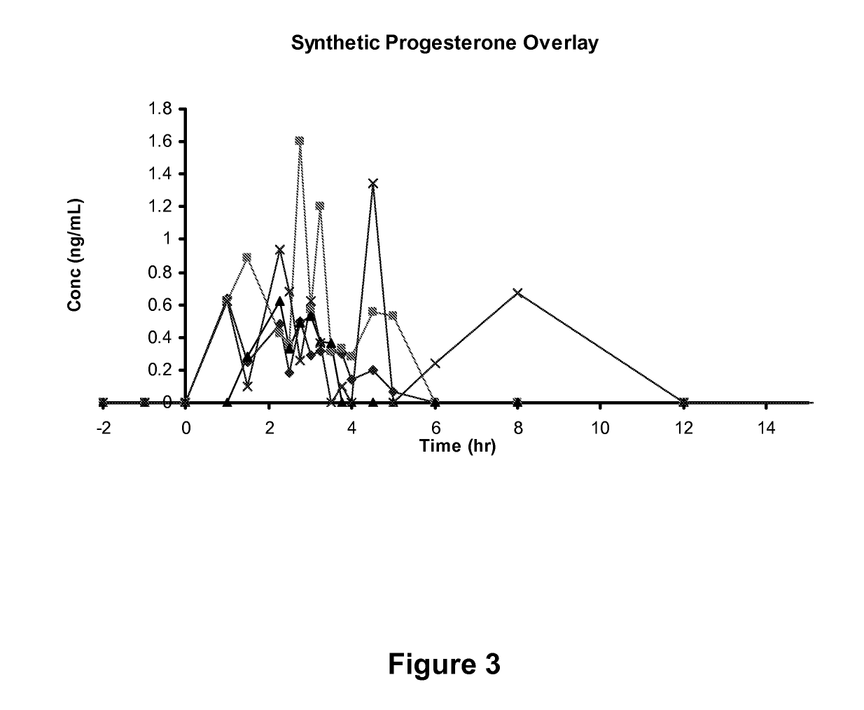

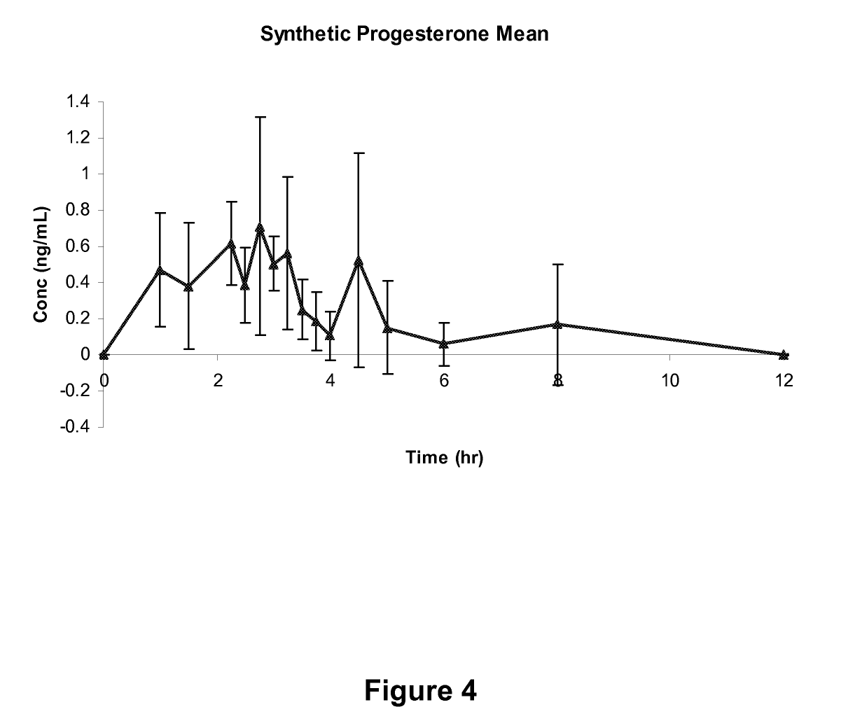

An overlay plot of progesterone time course for the four subjects is provided in FIG. 3 (synthetic progesterone) and a corresponding average and statistical parameter of those data is provided in FIG. 4.

### Example 3

### Pharmacokinetic (PK) Analysis

In this example, healthy, fasted, post-menopausal women orally ingest 1×200 mg progesterone. Plasma samples are obtained from 2 h pre-dose to 24 h post-dose. Analytes include total progesterone and synthetic progesterone, with a limit of quantification (LOQ) of 0.1 ng/mL. LOQ may be further reduced by varying one or more system parameters, such as to achieve an LOQ that is 0.01 ng/mL or better.

RESULTS: Six subjects are enrolled, received the test article and provided plasma samples for analysis. Total progesterone and synthetic progesterone concentrations are measured and reported in four subjects, with a quantifiable bioassay signal not being reported in the other two subjects. Pharmacokinetic parameters are determined in the four subjects with complete data sets using non-compartmental analysis. Parameters are determined from individual plasma concentration versus time data for total progesterone, synthetic progesterone and endogenous progesterone. Endogenous progesterone is calculated as the difference between total and synthetic progesterone. Individual and summarized results are presented in the following tables and figures.

PK Parameter calculations: Cₚᵣₑ is determined as the mean of the three plasma concentrations prior to dose administration (−2, −1 and 0 h samples). Cₘₐₓ is the maximum observed concentration and Tₘₐₓ the time at which Cₘₐₓ took place. Cₗₐₛₜ is the value of the last measurable concentration, and Tₘₐₓ the time at which Cₘₐₓ is observed. The area under the plasma concentration vs. time curve (“AUG”) is determined by linear trapezoidal integration from time zero to 4 h (AUC0-4 h) and from zero to Tₗₐₛₜ (AUCₗₐₛₜ). Concentrations reported as below the limit of quantitation (<0.1 ng/mL) are assigned a value of 0.0 for the pharmacokinetic analysis. The calculated value of endogenous progesterone at 3.5 h for subject 4 (−0.07 ng/mL) is also assigned a value of 0.0 for the analysis.

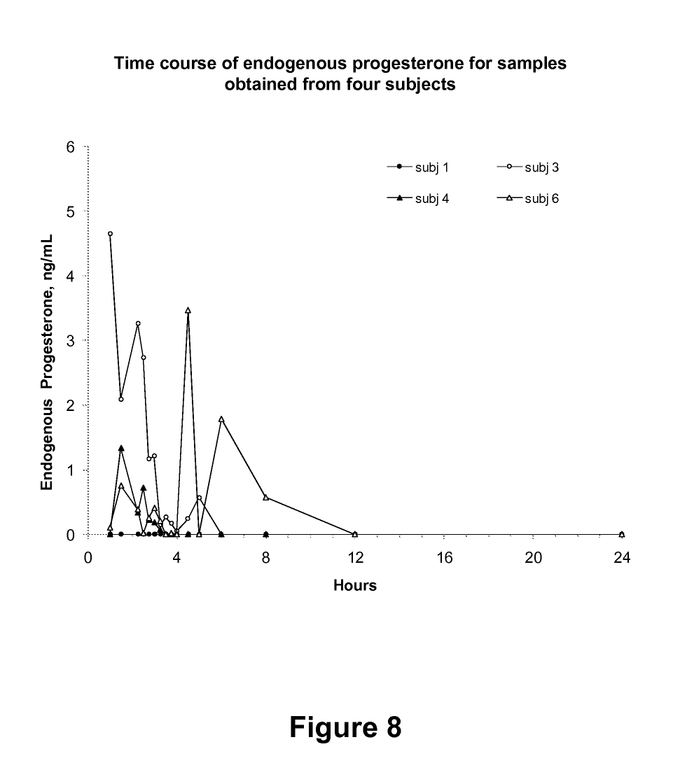

SUMMARY: Total and synthetic progesterone levels are below quantitation (<0.1 ng/mL) at all time points (−2, −1 and 0 h) prior to oral administration of progesterone 200 mg in the 4 subjects evaluated in the PK analysis. Referring to FIG. 2, following administration of progesterone 200 mg, total and synthetic progesterone levels rose in all 4 subjects, reaching maximum levels between 1 and 4.5 hours after administration. Cₘₐₓ ranged from 0.64 to 5.28 ng/mL for total progesterone, from 0.62 to 1.60 ng/mL for synthetic progesterone and from 0.0 to 4.65 ng/mL (FIG. 8) for endogenous progesterone. Concentrations persisted for a few hours (Tₗₐₛₜ ranged from 3.25 to 8 h) but then fell to unquantifiable levels (<0.1 ng/mL) at all time points after 8 h. Values for AUCₗₐₛₜ were similar to those for AUC0-4 h in all subjects, consistent with the observation that most of the exposure occurred in the first few hours after dosing. The AUC0-4 h showed considerable variability between subjects, ranging from 1.36 to 10.2 ng·h/mL for total progesterone, from 1.01 to 2.37 ng·h/mL for synthetic progesterone and from 0.0 to 7.85 ng·h/mL for endogenous progesterone. Given the degree of fluctuation observed in the plasma concentration vs. time curves, the half-life of progesterone in these subjects are not determined.

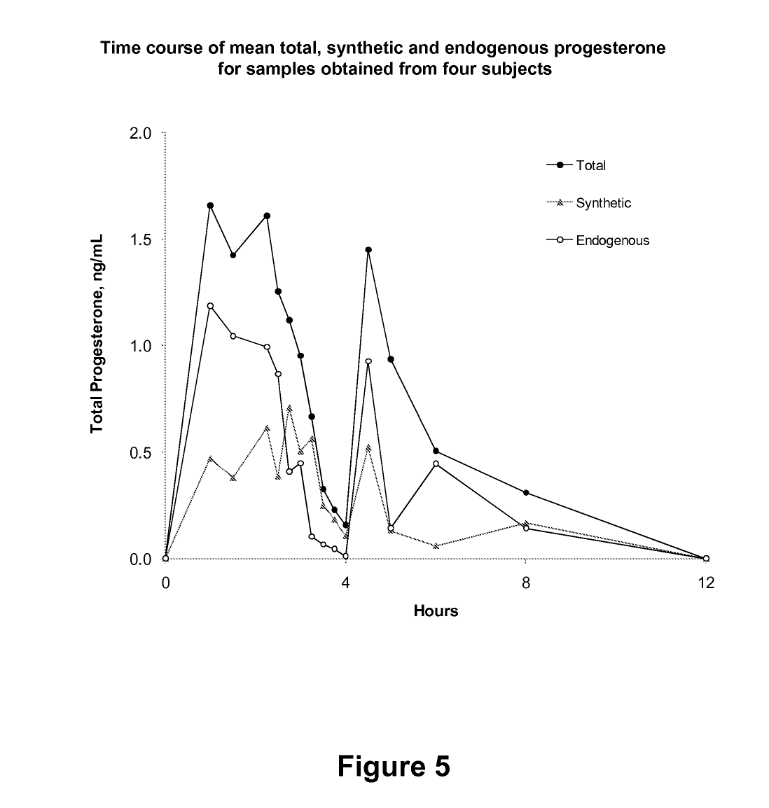

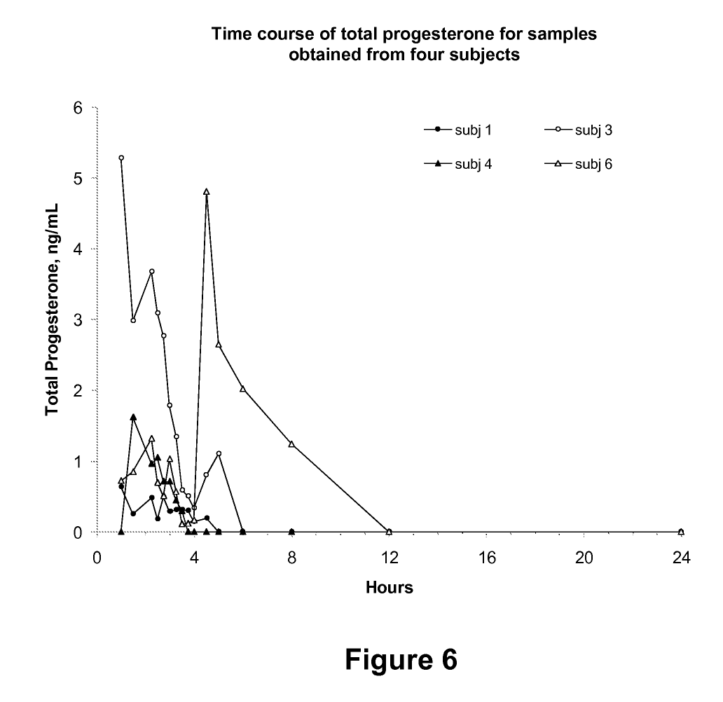

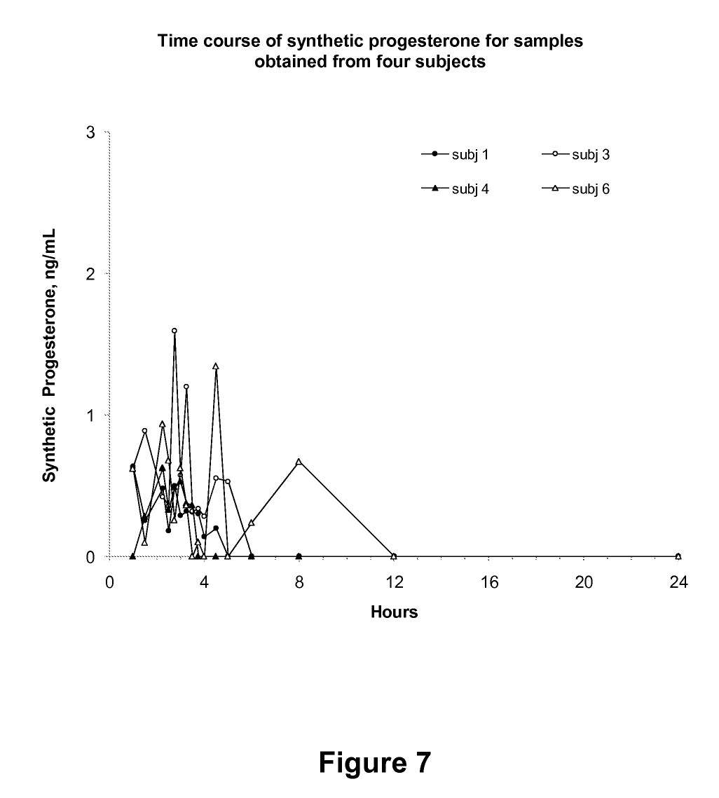

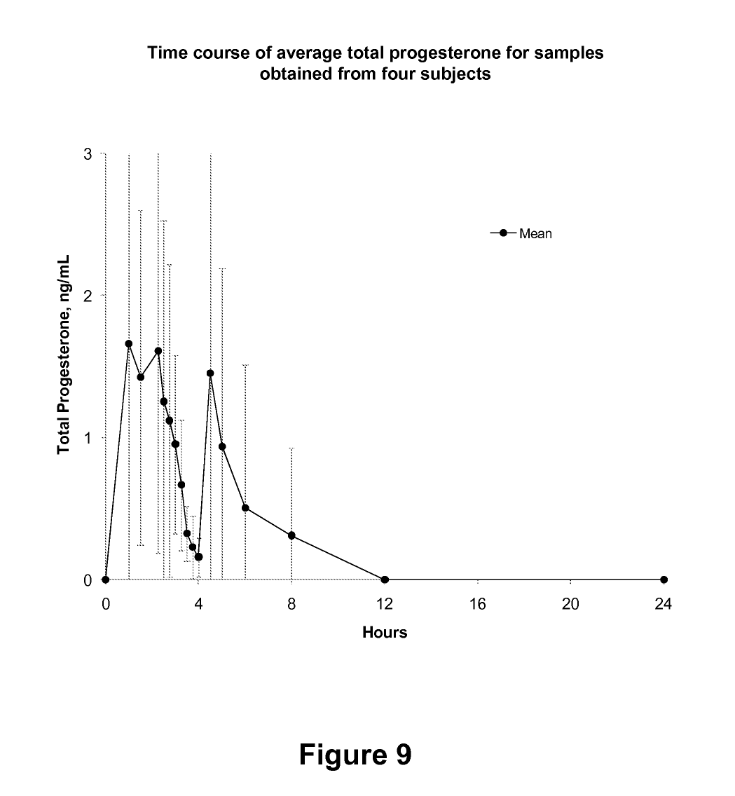

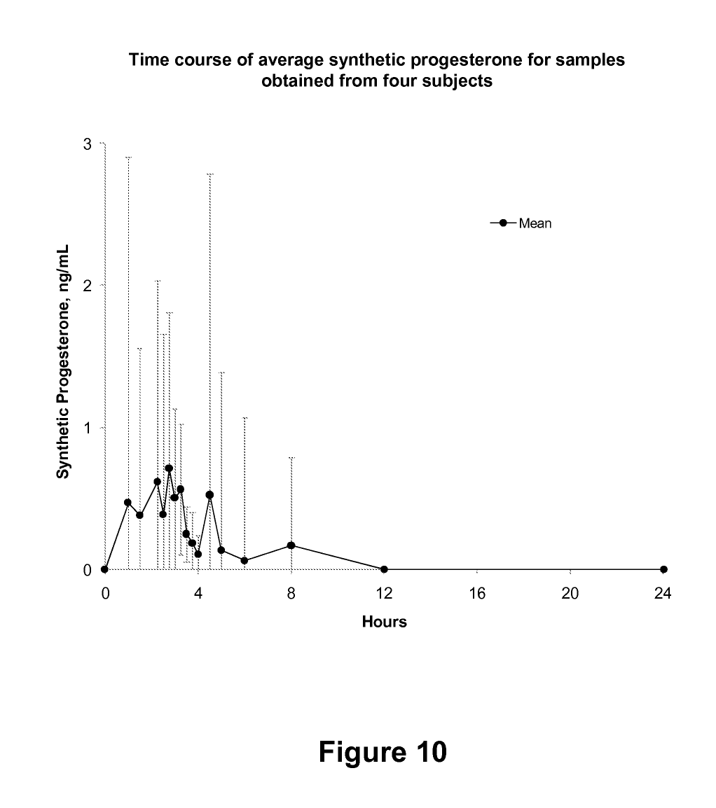

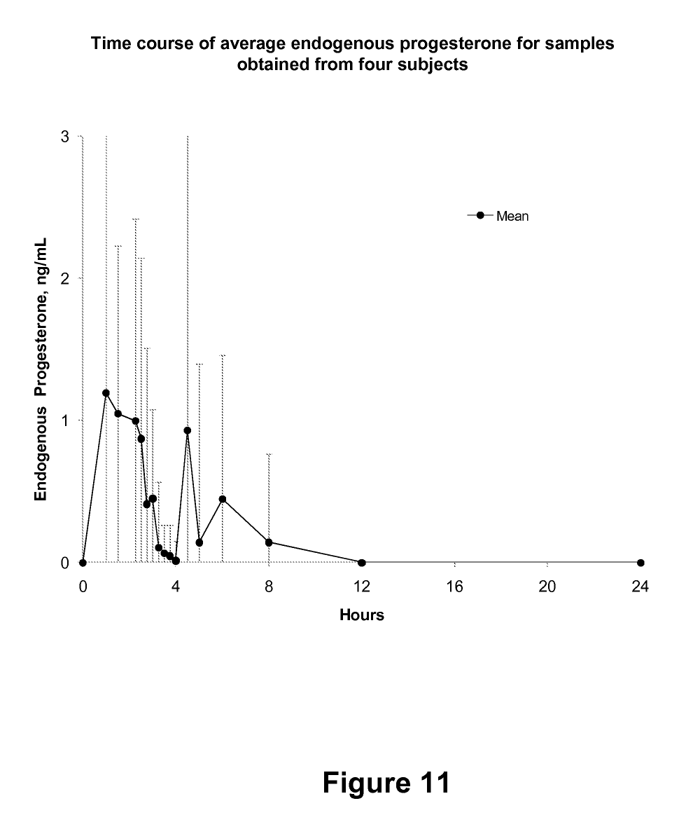

Plots of mean total, synthetic and endogenous progesterone concentrations exhibit broad peaks between about 1 and 3 h after administration, followed by a trough at 4 h and a secondary increase in progesterone concentrations at approximately 4.5 to 5 h (FIG. 5). However, individual concentration vs. time profiles show a high degree of variability over time and between subjects, making it difficult to define a clear concentration vs. time relationship in these subjects (FIGS. 6-8). However, for both total and synthetic progesterone, it should be noted that measurable levels were observed only during the 8-hour period following oral drug administration (all pre-dose, 12 h and 24 h samples were below quantitation), confirming that the progesterone detected and measured in this study occurred as a result of the administration of oral progesterone. Summary of PK parameters for oral administration of 200 mg progesterone is provided in TABLE 4 for each of total, synthetic and endogenous progesterone. Plots of PK time course, with the data averaged, for total, synthetic and endogenous progesterone are provided in FIGS. 9-11.

The ratio of synthetic progesterone to that of total progesterone varied considerably between the four subjects (Table 5). In one subject, synthetic progesterone accounted for all the progesterone measured (i.e., there was no endogenous progesterone). In the other subjects, synthetic progesterone accounted for approximately 26 to 43% of the total progesterone, based on Cₘₐₓ and AUC values for the two species. In these three subjects, synthetic progesterone levels appear to be lower than endogenous progesterone levels.

Overall these data suggest that subjects were exposed to progesterone over a period of several hours, as a result of a 200 mg oral dose, with significant inter-subject variability in concentrations, time course and ratio of synthetic to total progesterone.

Bioequivalence: One application of the methods provided herein relate to establishing bioequivalence of generic follow-on progesterone compounds. One reason for a lack of generic competition for progesterone is the difficulty in successfully completing a bioequivalence trial due to the high variability in progesterone plasma levels following oral dosing. A reason for high PK variability is due to changes in endogenous progesterone levels when synthetic progesterone is taken orally. Changes in progesterone level after application of synthetic progesterone associated with variations in endogenous progesterone may confound the statistical analysis. The development of an analytical technique that separates endogenous and synthetic progesterone, may reduce the coefficient of variance for key pharmacokinetic parameters for a given sample size. This reduction in variability results in a corresponding reduction in the number of patients required to establish bioequivalence. Currently, it is estimated that 440 patients per arm is required to obtain bioequivalence to a PROMETRIUM® progesterone.

Table 6 compares published pharmacokinetic parameters and corresponding statistical parameter data from the PROMETRIUM® progesterone package insert, to data generated using an analytical method disclosed herein that is capable of separating plasma levels of synthetic and endogenous progesterone. The package insert values uses a progesterone-quantification methodology that measures total progesterone (e.g., both synthetic and endogenous). As expected, the methodology disclosed herein can significantly reduce the variability of a statistical parameter (in this example, the coefficient of variation) for the PK parameter for synthetic progesterone. Not surprisingly, the absolute values of the PK are significantly lower for the measured values compared to those obtained from the package insert as the package insert values are from subjects administered five daily doses, in comparison to the single one-day dose used in the examples presented herein.

As seen from the data summarized in TABLE 6, coefficient of variance for three PK parameters (Cₘₐₓ, Tₘₐₓ, and AUC) is reasonably consistent between package insert published data and TOLMAR's total progesterone data (compare “total” against package insert values). Cmax varies by about 100%, Tmax varies by about 50%, and AUC varies by about 80%. Separating the fraction of plasma progesterone into endogenous and synthetic, i.e. plasma progesterone that came from the oral capsule, reduces the coefficient of variance by about 50% for Cmax and AUC. As expected Tmax variability appears to not be highly impacted by this improved analytical technique.

These data indicate that the analytical method disclosed herein is capable of quantitatively distinguishing between endogenous and synthetic progesterone. Some of the PK variability characteristic of oral progesterone dosing is due to the orally dosed synthetic progesterone altering (up-regulating) endogenous progesterone production in some post menopausal women. This example indicates that by separately quantifying synthetic progesterone from endogenous progesterone, a successful PROMETRIUM® progesterone bioequivalence PK study is estimated to require fewer patients per crossover arm. Such a reduction provides significant time and cost savings for regulatory studies to establish bioequivalence. A statistical power analysis (at 90% power for a two arm crossover bioequivalence study) indicates that replacing total progesterone with synthetic progesterone in the PK analysis (thereby decreasing the coefficient of variation in AUC from 99% to 47%—compare, e.g., Table 6 99% coefficient of variation for AUC from package insert for PROMETRIUM® 200 mg against 47% using a process disclosed herein) reduces the number of subjects per crossover arm from 224 to 51 to establish bioequivalence with PROMETRIUM®.

### REFERENCES

1. U.S. Pat. No. 7,473,560, Soldin. “Steroid Hormone Analysis by Mass Spectrometry”
2. US. Pub. No. 2004/0235188, Soldin. “Thyroid Hormone Analysis by Mass Spectrometry”
3. U.S. Pat. No. 7,348,137, Caulfield et al. “Determination of Testosterone by Mass Spectrometry”
4. PCT Pub. No. WO 01/88548, Kao et al. “Adrenal Dysfunction”
5. Aguilera et al. “Screening urine for exogenous testosterone by isotope ratio mass spectrometric analysis of one pregnanediol and two androstanediols.” J Chromatogr B. 727(1-2):95-105 (1999).
6. Aguilera et al. “Detection of testosterone misuse: comparison of two chromatographic sample preparation methods for gas chromatographic-combustion/isotope ratio mass spectrometric analysis” J Chromatogr B. 687(1): 43-53 (1996).
7. Aguilera et al. “Performance characteristics of a carbon isotope ratio method for detecting doping with testosterone based on urine diols: Controls and athletes with elevated testosterone/epitestosterone ratios” Clinical Chem. 47(2):292-300 (2001).
8. Boudou et al. “Comparison of progesterone concentration determination by 12 non-isotopic immunoassays and gas chromatography/mass spectrometry in 99 human serum samples” Journal of Steroid Biochemistry and Molecular Biology. 78(1): 97-104 (2001).
9. De Brabander et al. “Phytosterols and anabolic agents versus designer drugs” Analytica Chimica Acta 586(1-2):49-56 (2007).
10.Godin et al. “Liquid chromatography combined with mass spectrometry for 13C isotopic analysis in life science research” Mass Spectrometry Reviews 26(6): 751-774 (2007).
11. Kawaguchi et al. “Miniaturized hollow fiber assisted liquid-phase microextraction and gas chromatography—mass spectrometry for the measurement of progesterone in human serum” Journal of Chromatography B. 877(3): 343-346 (2009).
12. Lichtfouse. “Compound-specific isotope analysis. Application to archaelogy, biomedical sciences, biosynthesis, environment, extraterrestrial chemistry, food science, forensic science, humic substances, microbiology, organic geochemistry, soil science and sport” Rapid Communications in Mass Spectrometry 14(15):1337-1344 (2000).
13. Saudan et al. “Urinary marker of oral pregnenolone administration” Steroids 70(3): 179-183 (2005).
14. Schanzer “Recent Advances in Doping Analysis” in Peng et al. “overall Biological Markers of Oral Testosterone Undecanoate Misuse” pp. 185-203 (1999).
15. Segura et al. “Recent progress in the detection of the administration of natural hormones: Special focus on Testosterone” J. Toxic.—Toxin Reviews. 18(2):125-144 (1999).
16. Siekmann et al. “Quantitative Mass Spectrometry in Clinical Chemistry” Mikrochim. Acta [Wien] II, 145-155 (1991).
17. Siekmann “Determination of steroid hormones by the use of isotope dilution—mass spectrometry: a definitive method in clinical chemistry” J Steroid Biochem. 11:117-23 (1979).
18. Tai et al. “Development and evaluation of a candidate reference measurement procedure for the determination of progesterone in human serum using isotope-dilution liquid chromatography/tandem mass spectrometry” Anal Chem. 78(18):6628-33 (2006).
19. Tai et al. “Development and evaluation of a candidate reference measurement procedure for the determination of testosterone in human serum using isotope dilution liquid chromatography/tandem mass spectrometry” Anal Bioanal Chem. 388(5-6):1087-94 (2007).
20. Thienpont et al. “Determination of reference method values by isotope dilution-gas chromotography/mass spectrometry : A five years' experience of two European Reference Laboratories” European journal of clinical chemistry and clinical biochemistry. 34(10): 853-860 (1996).
21. Turpeinen et al. “Determination of testosterone in serum by liquid chromatography-tandem mass spectrometry” Scand J Clin Lab Invest. 68(1):50-7 (2008).
22. Wudy et al. “Determination of 17-hydroxyprogesterone in plasma by stable isotope dilution/benchtop liquid chromatography-tandem mass spectrometry.” Horm Res. 53(2):68-71 (2000).

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art.

When a Markush group or other grouping is used herein, all individual members of the group and all combinations and subcombinations possible of the group are intended to be individually included in the disclosure.

As used herein, “comprising” is synonymous with “including,” “containing,” or “characterized by,” and is inclusive or open-ended and does not exclude additional, unrecited elements or method steps. As used herein, “consisting of excludes any element, step, or ingredient not specified in the claim element. As used herein, “consisting essentially of does not exclude materials or steps that do not materially affect the basic and novel characteristics of the claim. Any recitation herein of the term “comprising”, particularly in a description of components of a composition, in a description of elements of a device or of a method step, is understood to encompass those compositions and methods consisting essentially of and consisting of the recited components or elements. The invention illustratively described herein suitably may be practiced in the absence of any element or elements, limitation or limitations which is not specifically disclosed herein.

Whenever a range is given in the specification, for example, a quantification limit, reduction range, improvement range, concentration range, sample size range, or a composition range, all intermediate ranges and subranges, as well as all individual values included in the ranges given are intended to be included in the disclosure.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The definitions provided herein are to clarify their specific use in the context of the invention.

TABLE 1
Column and MS Conditions
HPLC Conditions

| | |
|---|---|
| Injection Volume | 10 μL |
| Flow Rate (gradient) | 0.3 mL/min |
| Pump Gradient | See Table 7 |
| Eluants | A - 1% formic acid; B - 1% formic acid in ACN |
| Column | Waters 2.1X 250 mm, XBridge BEH130 C18 3.5 μm |
| Column Temperature | Ambient |
| Autosampler Temperature | 4° C. ± X° C. |

MS Conditions

| Mode | TurboSpray positive ionization | | | | | |
|---|---|---|---|---|---|---|
| Scan Type | Multiple Reaction Monitoring (MRM) | | | | | |
| Analysis Time | 23 minutes | | | | | |
| Analyte | Progesterone | | Progesterone Isotope | | 17α-ethynlestradiol (IS) | |
| Ion Transition | 315.0 → 109.0 amu | | 85.0 → 67.1 amu | | 283.0 → 135.0 amu | |
| Curtain Gas | 30 | L/min | 30 | L/min | 30 | L/min |
| Nebulizer Current | 5.00 | volts | 5.00 | volts | 5.00 | volts |
| Collision Gas | 11 | L/min | 11 | L/min | 11 | L/min |
| Temperature | 400° | C. | 400° | C. | 400° | C. |
| Ion source Gas 1 | 60 | L/min | 60 | L/min | 60 | L/min |
| Declustering Potential | 46 | volts | 46 | volts | 46 | volts |
| Collision Cell Exit Potential | 2.0 | volts | 2.0 | volts | 2.0 | volts |
| Collision Energy | 37 | volts | 33 | volts | 27 | volts |
| Collision Cell Entrance Potential | 10 | volts | 10 | volts | 10 | volts |

TABLE 2
Pump Gradient

| Time | % B |
|---|---|
| Initial | 30 |
| 15 | 60 |
| 15.1 | 90 |
| 17 | 90 |
| 17.1 | 30 |
| 23.1 | Stop |

TABLE 3
Calibration Solutions Preparation

| | Volume of | Progesterone | Concentration of | Volume of | Final Progesterone |
|---|---|---|---|---|---|
| Calibration | Progesterone Standard | Dilution | Progesterone Standard | IS DS A | Concentration |
| Solution | (uL) | Standard | (ng/mL) | (uL) | (ng/mL) |
| 1 | 20 | B | 50 | 300 | 0.1 |
| 2 | 100 | B | 50 | 300 | 0.5 |
| 3 | 200 | B | 50 | 300 | 1.0 |
| 4 | 1000 | B | 50 | 300 | 5.0 |
| 5 | 300 | A | 500 | 300 | 15.0 |
| 6 | 500 | A | 500 | 300 | 25.0 |
| 7 | 800 | A | 500 | 300 | 40.0 |
| 8 | 1000 | A | 500 | 300 | 50.0 |

TABLE 4
Summary PK Parameters for oral Progesterone 200 mg.

| | | | | |
|---|---|---|---|---|
| Mean | SD | cv | min | max |

Total Progesterone

| | | | | | | |
|---|---|---|---|---|---|---|
| Cpre | ng/mL | 0.00 | 0.00 | ND | 0.00 | 0.00 |
| Cmax | ng/mL | 3.09 | 2.31 | 75 | 0.64 | 5.28 |
| Tmax | h | 2.00 | 1.68 | 84 | 1.0 | 4.5 |
| Clast | ng/mL | 0.71 | 0.54 | 76 | 0.20 | 1.24 |
| Tlast | h | 5.25 | 1.94 | 37 | 3.5 | 8 |
| AUC0-4 h | ng h/mL | 4.10 | 4.11 | 100 | 1.36 | 10.2 |
| AUClast | ng h/mL | 6.47 | 5.35 | 83 | 1.45 | 11.2 |

Synthetic Progesterone

| | | | | | | |
|---|---|---|---|---|---|---|
| Cpre | ng/mL | 0 | 0 | ND | 0 | 0 |
| Cmax | ng/mL | 1.05 | 0.50 | 47 | 0.624 | 1.60 |
| Tmax | h | 2.63 | 1.45 | 55 | 1.0 | 4.5 |
| Clast | ng/mL | 0.44 | 0.21 | 47 | 0.196 | 0.670 |
| Tlast | h | 5.25 | 1.94 | 37 | 3.5 | 8 |
| AUC0-4 h | ng h/mL | 1.56 | 0.58 | 37 | 1.01 | 2.37 |
| AUClast | ng h/mL | 2.11 | 1.08 | 51 | 0.96 | 3.20 |

Endogenous Progesterone

| | | | | | | |
|---|---|---|---|---|---|---|
| Cpre | ng/mL | 0.0 | 0.0 | ND | 0.0 | 0.0 |
| Cmax | ng/mL | 2.36 | 2.09 | 88 | 0.0 | 4.65 |
| Tmax | h | 2.33^{a} | 1.89 | 81 | 1.0 | 4.5 |
| Clast | ng/mL | 0.30 | 0.31 | 103 | 0.0 | 0.57 |
| Tlast | h | 5.42^{a} | 2.40 | 44 | 3.25 | 8 |
| AUC0-4 h | ng h/mL | 2.53 | 3.59 | 142 | 0.00 | 7.85 |
| AUClast | ng h/mL | 3.84 | 3.83 | 100 | 0.00 | 8.13 |

N = 4 unless noted
ND, Not determined
^{a}N = 3.

TABLE 5
Ratios of synthetic to total progesterone

| Subject | “1” | “3” | “4” | “6” | MEAN | SD | min | max |
|---|---|---|---|---|---|---|---|---|
| Cmax, | 1.0 | 0.30 | 0.39 | 0.28 | 0.49 | 0.34 | 0.28 | 1.0 |
| synth/total | | | | | | | | |
| AUC, | 1.0 | 0.26 | 0.43 | 0.29 | 0.49 | 0.35 | 0.26 | 1.0 |
| synth/total | | | | | | | | |

TABLE 6
PK parameter comparison obtained from the PROMETRIUM ® package
insert and those obtained by a method of the present invention
after a single 200 mg dose of PROMETRIUM ®.

| | PROMETRIUM after 5 daily doses | | | PROMETRIUM 200 mg | |
|---|---|---|---|---|---|
| | (From package insert) | | | (Single dose, n = 4) | |
| | 100 mg | 200 mg | 300 mg | total | synthetic |
| Cmax | 17.3 + 21.9 | 38.1 + 37.8 | 60.6 + 72.5 | 3.1 + 2.3 | 1.05 + 0.5 |
| cv | 126% | 99% | 120% | 75% | 47% |
| Tmax | 1.5 + 0.8 | 2.3 + 1.4 | 1.7 + 0.6 | 2.0 + 1.68 | 2.6 + 1.4 |
| cv | 53% | 61% | 35% | 84% | 55% |
| AUC | 43.3 + 30.8 | 101.2 + 66.) | 175.7 + 170.3 | 4.1 + 4.1 | 1.6 + 0.6 |
| cv | 71% | 65% | 97% | 100% | 37% |

We claim:

1. A method of measuring a progesterone analyte in a blood fluid sample, said method comprising the steps of:
providing the blood fluid sample;
introducing a progesterone component obtained from said sample to a mass spectrometer;
measuring a carbon isotope ratio of said progesterone component; and
calculating from said isotope ratio a fraction of synthetic progesterone in said introduced progesterone component, thereby measuring said progesterone analyte in said sample.

2. The method of claim 1, further comprising:
obtaining said sample from a subject; and
isolating said progesterone component from said sample.

3. The method of claim 1, wherein at least any two of synthetic, endogenous, and total progesterone are measured.

4. The method of claim 1, further comprising calculating a concentration or amount of said progesterone analyte in said sample.

5. The method of claim 4, further comprising calculating a concentration or amount of endogenous progesterone in said sample.

6. The method of claim 1, further comprising isolating said progesterone component by liquid chromatography.

7. The method of claim 1, wherein said mass spectrometer is a liquid chromatography-tandem mass spectrometer.

8. The method of claim 1, wherein said blood fluid sample is plasma, serum or whole blood.

9. The method of claim 1, wherein said sample is obtained from a human.

10. The method of claim 1, further comprising administering synthetic progesterone to an individual prior to obtaining said blood fluid sample, wherein said synthetic progesterone is derived from a plant source.

11. The method of claim 10, wherein said plant source is yam from the genus Dioscorea.

12. The method of claim 1, wherein said calculating step comprises quantification of one or more of synthetic progesterone, endogenous progesterone and total progesterone, wherein the quantification is capable of detecting synthetic progesterone, endogenous progesterone or total progesterone at a level that is:
less than or equal to 0.1 or 0.01 ng/mL; or
from about 0.01 ng/mL to 0.1 ng/mL.

13. The method of claim 1, further comprising generating a carbon isotope ratio curve or equation that provides a fraction of synthetic or endogenous progesterone for a measured ¹³C/¹²C isotope ratio for a defined fraction of synthetic progesterone in a progesterone-containing sample.

14. The method of claim 1, wherein said calculating step comprises:
calculating the fraction of synthetic progesterone in said sample by providing a carbon isotope ratio curve or equation that defines the fraction of synthetic progesterone for the measured progesterone isotope ratio; and
calculating a synthetic progesterone level from said fraction.

15. A method of quantifying a progesterone analyte in a subject, said method comprising:
optionally providing said subject with progesterone;
obtaining a blood fluid sample from said subject;
isolating a progesterone component from said sample;
introducing said progesterone component to a mass spectrometer;
measuring a carbon isotope ratio of said progesterone component; and
calculating from said isotope ratio the amount of progesterone analyte in said sample, thereby quantifying the progesterone analyte in the subject.

16. The method of claim 15, further comprising:
repeating said method for a plurality of subjects;
calculating a pharmacokinetic parameter for said plurality of subjects from said measured isotope ratios; and
calculating a statistical parameter for said pharmacokinetic parameter.

17. The method of claim 16, wherein said statistical parameter is reduced compared to a corresponding statistical parameter calculated using a conventional progesterone quantifying method.

18. The method of claim 17, wherein said reduction is by at least 20%, at least 50%, or from about 20% to 80%.

19. The method of claim 17, wherein said statistical parameter is a coefficient of variation, standard deviation, standard error of the mean, or a range.

20. The method of claim 17, wherein said pharmacokinetic parameter is selected from the group consisting of:
Cₘₐₓ;
Tₘₐₓ;
half life; and
AUC.

21. The method of claim 15, wherein said provided progesterone results in an increase in endogenous progesterone in said sample.

22. A method of evaluating bioequivalence of a synthetic progesterone composition, said method comprising the steps of:
administering said composition to a plurality of subjects;
obtaining a blood fluid sample from said subjects after said administering step;
quantifying synthetic progesterone in said sample by measuring a carbon progesterone isotope ratio; and
calculating a synthetic progesterone pharmacokinetic parameter from said isotope ratio.

23. The method of claim 22, wherein said bioequivalence is evaluated by comparing said calculated pharmacokinetic parameter against a corresponding pharmacokinetic parameter from a second synthetic progesterone-containing compound, said corresponding pharmacokinetic parameter is obtained from a publication or using a method disclosed herein.

24. The method of claim 23, wherein said pharmacokinetic parameter is one or more of Cₚᵣₑ, Cₘₐₓ, Tₘₐₓ, Cₗₐₛₜ and AUC.

25. The method of claim 22, wherein bioequivalence is evaluated using a subject number that is less than the number required using a conventional progesterone-quantifying assay that does not distinguish between synthetic and endogenous progesterone.

26. The method of claim 25, wherein the subject number is at least 20% less than, or at least 50% less than the number required using a conventional progesterone-quantifying assay.

27. The method of claim 25, wherein the subject number for evaluating bioequivalence is selected from the group consisting of:
less than 400;
less than 300; and
less than 250.

28. The method of claim 22, further comprising:
calculating a statistical parameter for said pharmacokinetic parameter;
wherein said statistical parameter is reduced by at least 20% compared to a corresponding statistical parameter obtained using a conventional progesterone-quantifying assay that does not distinguish between synthetic and endogenous progesterone.

29. The method of claim 28, wherein said statistical parameter is standard deviation, standard error of the mean, coefficient of variation, or a range.

30. The method of claim 22, wherein said sample is obtained between 1 hour and 8 hours after said synthetic progesterone administration step.

31. The use of the method of claim 22 to evaluate bioequivalence of one synthetic progesterone-containing compound to a second synthetic progesterone-containing compound.

32. The method of claim 22, wherein the synthetic progesterone is PROMETRUIM® progesterone (pregn-4-ene-3,20-dione) by Solvay Pharmaceuticals, Inc. (Marietta, Ga.).

33. The method of claim 1, wherein the progesterone analyte corresponds to synthetic progesterone.

34. The method of claim 1, wherein the progesterone component comprises synthetic and endogenous progesterone.

35. The method of claim 1, wherein the carbon isotope ratio is the ratio of ¹³C to ¹²C.

36. The method of claim 1, wherein the sample is from a subject that is fasted.

37. The method of claim 1, wherein the sample is from a subject that is fed.

38. The method of claim 1, wherein the sample is from a post-menopausal individual.

39. The method of claim 1, wherein the sample is from a female.

40. A kit for measuring a progesterone analyte, comprising a set of at least two reference samples with varying carbon isotope ratios of plant source progesterone to animal source progesterone.

41. The kit of claim 40, wherein the set comprises at least seven reference samples and wherein at least two of said samples comprise a detectable amount of human plasma.
